(19) United States
(12) Patent Application Publication
(10) Pub. No.: US 2020/0113613 A1
(43) Pub. Date: Apr. 16, 2020

(54) CRYO NEEDLE GUIDE

(71) Applicant: EIGEN HEALTH SERVICES LLC, GRASS VALLEY, CA (US)

(72) Inventors: PABLO MEDINA, Marysville, CA (US); RAJESH VENKATARAMAN, ROCKLIN, CA (US); BRADLEY MARTINEZ, SACRAMENTO, CA (US); YANG XU, NEVADA CITY, CA (US)

(21) Appl. No.: 16/599,938

(22) Filed: Oct. 11, 2019

Related U.S. Application Data
(60) Provisional application No. 62/744,849, filed on Oct. 12, 2018.

(51) Int. Cl. A61B 18/02; A61B 18/14; A61B 8/00

(52) U.S. Cl. CPC A61B 18/02; A61B 18/1477; A61B 18/1482; A61B 2017/3413; A61B 8/461; A61B 2018/0262; A61B 2018/0293; A61B 8/4444

(57) ABSTRACT

A needle guidance assembly for constraining and guiding an interventional needle is provided. The needle guidance assembly includes a needle guide having opposing lever arms. Jaws of the opposing lever arms form a guide bore configured to receive an interventional needle and orient the needle with respect to, for example, an imaging probe. Constraining the needle within the imaging field of the probe allows for real-time monitoring during insertion of the interventional needle into patient tissue. In addition, the needle guidance assembly is angularly positionable relative to the image field to allow advancement of the needle to a target site within the imaging field.

### CROSS REFERENCE

The present application claims the benefit of the filing date of U.S. Provisional Application No. 62/744,849 having a filing date of Oct. 12, 2018, the entire contents of which is incorporated herein by reference.

### FIELD

The present disclosure is directed to utilities (i.e., systems, methods and apparatuses) for guiding interventional needles during medical procedures. More particularly, the disclosure relates to utilities that allow for guiding and maintaining an interventional needle in a fixed relationship with a medical imaging instrument.

### BACKGROUND

Doctors and other medical professionals often utilize medical imaging instruments to conduct non-invasive examinations. That is, medical imaging instruments, including X-ray, magnetic resonance (MR), computed tomography (CT), ultrasound, and various combinations of these instruments/techniques, are utilized to provide images of internal patient structure for diagnostic purposes as well as for interventional procedures. Such medical imaging instruments allow examination of internal tissue that is not readily examined during normal visual or tactile examination, which could then be used for either diagnosis (e.g. MRI for prostate) or for guidance to a region of interest in the body (e.g. interventional procedures like biopsies, therapy, etc.).

Medical imaging instruments typically allow for generating three-dimensional (“3D”) images of internal structures of interest, often by interleaving a series of 2D images. For instance, a medical imaging device may be utilized to generate a 3D model or map of the prostate such that one or more biopsies may be taken from certain desired locations of the prostate and/or therapy may be delivered to those desired locations of the prostate. For purposes of prostate imaging, a transrectal ultrasound-imaging device (TRUS) provides image acquisition and guidance. TRUS probe is the most widely accepted technique for prostate applications due to its simplicity, high specificity, and real time nature. In such an application, the TRUS probe or similar medical imaging device may be inserted into the rectum of a patient to generate one or more 2D images. Such images may be utilized to generate a 3D image of the prostate that may subsequently be utilized to take one or more biopsies from a prostate location of interest and/or apply therapy (e.g., implant radioactive seeds) at one or more desired locations.

For procedures that require precision, such as targeted biopsy and other treatment procedures, it is desirable that the relative location between an imaging instrument and an anatomical area of interest be known. That is, it is important that the image plane of a medical imaging instrument covers a particular tissue location and remains stationary to allow for guiding a biopsy/treatment device to that tissue location within the imaging field. Relative movement between the imaging device and the tissue area of interest during imaging and/or biopsy/treatment may impede the successful performance of these procedures. Accordingly, a number of holding and manipulating/positioning assemblies have been proposed wherein a holder interfaces with an imaging device such as a TRUS probe. The holder may be interconnected to one or more mechanical armatures and/or actuators such that the probe may be precisely controlled and mechanically positioned and/or rotated relative to an area of interest on a patient (a “tracking assembly”) to maintain a fixed position relative to the patient.

Similarly, it is critical for interventional procedures that the medical imaging instrument is also maintained in fixed relation to the interventional needle. In this regard, a needle guide may be affixed to the holder to direct the interventional needle to a desired location within the image plane of the probe. U.S. patent application Ser. No. 15/203,417 describes an embodiment of one such needle guide. However, such guides may be cumbersome and there remains a need for a needle guide that may be quickly deployed to simplify the process of introducing an interventional needle during a medical procedure.

### SUMMARY

Provided herein are utilities (i.e., apparatuses, systems and methods) that combine the positioning and support of a needle guidance assembly with respect to a medical imaging instrument (e.g., ultrasound probe) such that an interventional needle (e.g., therapy delivery device, biopsy needle, trocar, etc.) held by the needle guidance assembly, for insertion into patient tissue, is constrained within an imaging field of the medical imaging instrument. Constraining the biopsy treatment device within the imaging field allows for real-time monitoring of the biopsy/treatment device during insertion into patient tissue. In addition, the needle guidance assembly is angularly positionable relative to the imaging field to allow advancement of the interventional needle to any desired location within the imaging field. The interventional needle may be used to take biopsies and/or apply therapeutic matter such as, for example, brachytherapy seeds, cryoablation fluid (e.g., liquid or gas), ablation energy, and/or electroporation energy (electric field energy). In one arrangement, movement of the needle guidance assembly is limited to a single degree of freedom allowing angular positioning of an interventional needle within a two-dimensional image plane.

According to a first aspect, a needle guide for medical diagnoses and treatment includes first and second lever arms, a pivot pin, and a guide bore. The pivot pin may connect the first and second lever arms and define a pivot point therebetween. Each of the lever arms may include a jaw and a handle extending from the jaw on an opposing side of the pivot point. The first and second lever arms may have a closed configuration in which the jaws are in physical contact with one another and the handles are spaced apart. (e.g., by a maximum distance). The lever arms may also have an open configuration in which the jaws are spaced apart and the handles are spaced apart by less than the maximum distance. The guide bore may be configured to receive an interventional needle. The guide bore may be defined by the jaws when in the closed configuration.

In an embodiment, a first C-shaped channel along the length of the jaw of the first lever arm may mirror a corresponding second C-shaped channel along the length of the jaw of the second lever arm. In this regard, the first and second C-shaped channels may form the guide bore when in the closed configuration. The first and second C-shaped channels may be contiguous in the closed configuration and completely enclose the guide bore. Alternatively, at least a portion of the first and second C-shaped channels may be spaced apart in the closed configuration such that a portion of the guide bore is unenclosed by the first and second C-shaped channels.

In another embodiment, a needle guide may include an optional biasing mechanism configured to bias the first and second lever arms toward the closed configuration. Such a biasing mechanism may be a spring in compression disposed between the handle of the first lever arm and the handle of the second lever arm or may be a spring in tension disposed between the jaw of the first lever arm and the jaw of the second lever arm.

In yet another embodiment, a needle guide may include a mounting bracket. The first and second lever arms may be affixed to the mounting bracket. The mounting bracket may be configured for removable attachment to a base member of a system configured to hold a medical imaging instrument in pivotal relation to the base member. One or both lever arms may be affixed to the mounting bracket via the pivot pin.

In another aspect, a system for medical diagnoses and treatment may include a probe holder, an interventional needle, and a needle guidance assembly. The probe holder may be configured to hold a medical imaging instrument. The needle guidance assembly may include a base member and a needle guide. The base member may be pivotally attached to the probe holder for angular manipulation of the needle guidance assembly with respect to the medical imaging instrument. The needle guide may be removably attachable to the base member to retain the needle guide in fixed relation to the base member. The needle guide may include lever arms, a pivot pin, and a guide bore as described above. A trajectory axis of the guide bore may be aligned within an image plane of the medical imaging instrument when the medical imaging instrument is disposed within the probe holder.

The probe holder may generally form a recessed surface or cradle configured to receive and secure a portion of a medical imaging instrument. In such an arrangement, an acquisition portion (e.g., transducer array) of the medical imaging instrument is secured in a known relationship to the probe holder. The probe holder may include a rotatable coupling adapted for rotatable connection with a positioning device such that the probe holder and supported medical imaging instrument are operative to rotate about a rotational axis of the positioning device. The positioning device may include various encoders that output a 3D position and/or orientation of the attached probe holder and supported medical imaging instrument. As a fixed relation of an acquisition portion of the medical imaging instrument is known relative to the probe holder, the orientation of the acquisition portion is known in a 3D space of the positioning device. This allows for locating images from the medical imaging instrument in the known 3D space. In one arrangement, an acquisition axis of an ultrasound probe is aligned with the rotational axis of the positioning device.

In addition, the system includes a needle guidance assembly having a guide bore (e.g., needle guide bore) that may be aligned with an image plane of a medical imaging instrument when instrument is secured within a probe holder. Thus, the spatial relationship of the needle guidance assembly is known relative to the acquisition axis or imaging field of the medical imaging instrument. In this regard, a trajectory (e.g., needle trajectory) of the guide bore may be plotted on an output image of the medical imaging instrument. Thus, the medical imaging instrument may be rotated to display a desired portion of an anatomical internal structure having, for example, a target site (e.g., prostate lesion). An image (e.g., 2D image from the image plane of the instrument) including the target site may be generated on a display. Further, the trajectory of the guide bore (e.g., needle trajectory) may be superimposed on the image. To permit alignment of the guide bore trajectory (and subsequently the interventional needle) with the target site, the needle guidance assembly may rotate relative to the probe holder to adjust the guide bore trajectory within the image plane. Thus, the guide bore trajectory may be aligned with a target site within the image plane. Accordingly, a user may extend an interventional needle through the guide bore of the needle guidance assembly into the patient and ultimately to the target site. Such insertion may be executed while monitoring real-time imaging.

In an embodiment, a medical imaging instrument may be a side-fire ultrasound probe. The base member may rotate about a second axis that is transverse to an image plane of the ultrasound probe.

In some embodiments, the interventional needle may be a biopsy needle configured to extract tissue samples or may be configured to deposit or apply therapeutic matter. For example, therapeutic matter may include at least one of: brachytherapy seeds; a cryoablation fluid (e.g., liquid, gas, plasma etc.); ablation energy; and electroporation energy.

In another aspect, a method of administering treatment is provided. The method may include, inter alia, scanning a patient with a medical imaging instrument disposed in a probe holder; identifying a target site within tissue of the patient; aligning a guide bore of a needle guidance assembly with the target site, and extending an interventional needle through the guide bore into the patient tissue to the target site. The needle guidance assembly may be similar to that described above.

### BRIEF DESCRIPTION OF THE DRAWINGS

FIG. 1A shows a cross-sectional view of a transrectal ultrasound imaging system as applied to perform prostate imaging.

FIG. 1B illustrates use of a positioning device to position an ultrasound imaging device to perform prostate imaging.

FIG. 2A illustrates two-dimensional images generated by a medical imaging instrument such as the transrectal ultrasound imaging system of FIG. 1.

FIG. 2B illustrates a three-dimensional volume image generated from the two-dimensional images of FIG. 2A.

FIG. 3 illustrates a prior art solution for needle guidance during biopsy or therapy.

FIG. 4A illustrates various views of a needle guide in accordance with the present disclosure in the closed configuration.

FIG. 4B illustrates various views of the needle guide of FIG. 4A in the open configuration.

FIG. 5A illustrates one embodiment of a probe holder with an ultrasound probe and a needle guidance assembly in accordance with the present disclosure.

FIG. 5B illustrates a front profile of the needle guidance assembly of FIG. 5A in relation to the ultrasound sound. alignment of a guide bore with an image plane with the embodiment of FIG. 5A.

FIG. 6 alignment of a guide bore with an image.

FIG. 7 illustrates an angular offset of the needle guidance assembly relative to the probe holder/cradle.

FIG. 8 illustrate a needle inserted in the needle guidance assembly of FIG. 7.

FIG. 9A illustrates a front view of the jaws of a needle guide having a fully enclosed guide bore.

FIG. 9B illustrates a front view of the jaws of a needle guide having a partially enclosed guide bore.

### DETAILED DESCRIPTION

Reference will now be made to the accompanying drawings, which assist in illustrating the various pertinent features of the present disclosure. Although described primarily in conjunction with transrectal ultrasound imaging for prostate imaging, biopsy, and therapy, it should be expressly understood that aspects of the present disclosure may be applicable to other medical imaging applications. In this regard, the following description is presented for purposes of illustration and should not be considered as limiting the scope of the invention.

Utilities are disclosed that facilitate obtaining medical images and/or performing medical procedures. One embodiment provides a combined medical imaging instrument holder (e.g., probe holder) and a needle guidance assembly. The needle guidance assembly maintains a trajectory of a supported interventional needle (that term is used herein to generally refer to any elongated medical device needing precise guidance, e.g., needle, trocar, therapy device, etc.), which is configured for insertion into patient tissue. The needle guide of the needle guidance assembly may be used independently, may be used with a base member for stabilizing the needle guide, or may be used with a probe holder to restrain the trajectory of the needle within an imaging field of a medical imaging instrument (e.g., two-dimensional image plane of an ultrasound probe) held by the probe holder. The probe holder may be configured for rotational attachment with a positioning device allowing the location of the medical imaging instrument and its image plane to be known in a 3D space. The positional relationship between the probe holder and the needle guidance assembly may be maintained while the probe holder is rotated. In this regard, the medical imaging instrument supported by a probe holder may obtain multiple 2D or 3D images of patient's anatomy in different orientations. The attached needle guidance assembly may be utilized to direct an interventional needle through the patient's tissue to a target site within an image plane of the medical imaging instrument. For example, a biopsy needle may be directed through the guide bore of a needle guidance assembly, through a patient's perineum, and into the patient's prostate to extract a tissue sample. As the trajectory of the needle is aligned within the image plane of the medical imaging instrument, the progression of the biopsy needle may be displayed on a real-time image of the imaging device such that targeting may be performed under real-time image guidance.

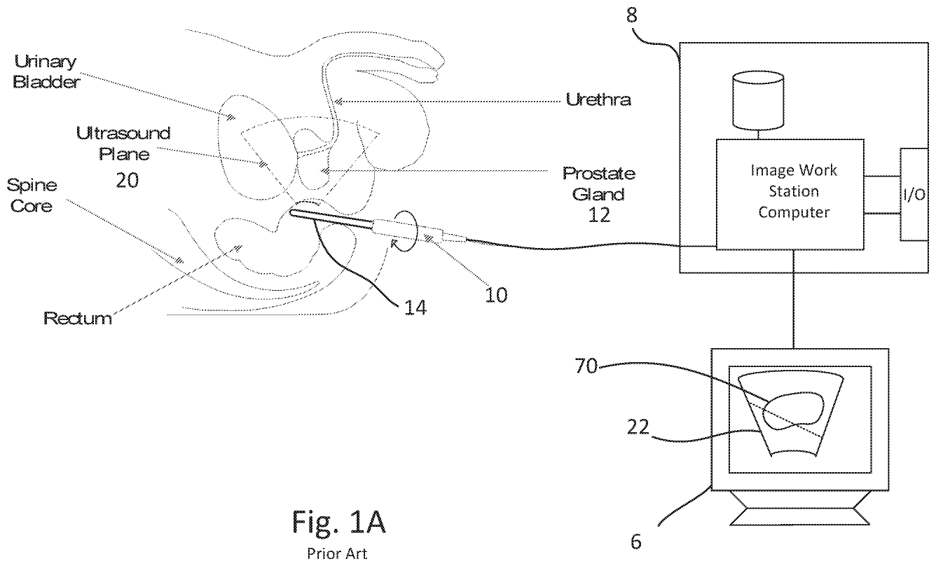

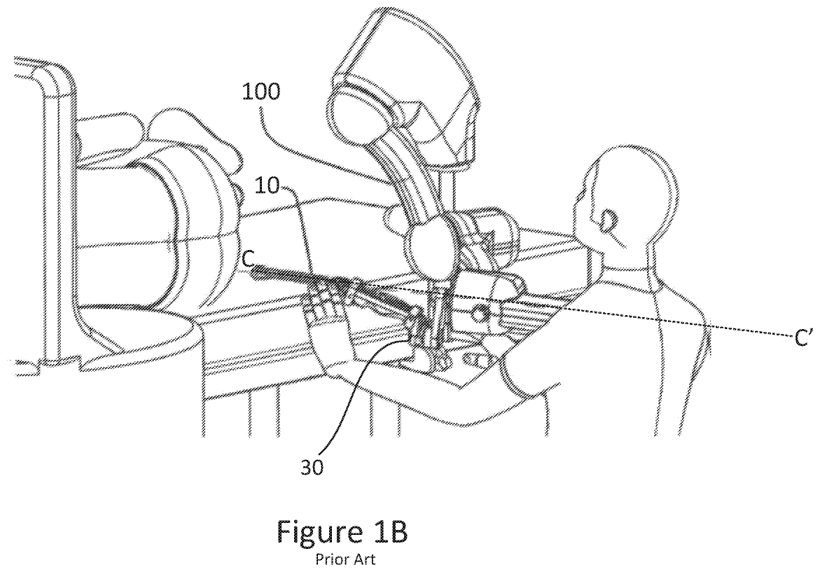

FIG. 1A illustrates a transrectal ultrasound probe 10 being utilized to obtain a plurality of 2D ultrasound images of the prostate 12. The illustrated probe 10 scans an area of interest along an image plane 20. In such an arrangement, a user may rotate the acquisition portion 14 of the ultrasound probe 10 across an area of interest. The image(s) 22 taken along the image plane 20 of the probe 10 are provided to an imaging system 8 and output to a display 6. The probe 10 may acquire a plurality of individual images while being rotated to capture the area of interest.

As shown in FIG. 1A, the ultrasound probe 10 is a side-fire probe that generates ultrasound waves out of a side surface of its acquisition portion 14 which is transverse to an acquisition axis. However, it is contemplated that other imaging devices (e.g., end-fire probes) may be used in other embodiments. The illustrated system can be used to generate a series of images 22 of the prostate 12 while the probe 10 is positioned relative to the prostate. If there is little or no movement between acquisition of the images, these images may be readily registered together to generate a 3D image as described below. However, manual manipulation of the probe 10 often results in relative and unaccounted movement between the probe 10 and the prostate 12 between subsequent images. Accordingly, it is desirable to minimize relative movement between the probe 10 and the prostate 12 (i.e., precession, wobble, or any other rotational movement of the probe about a fixed axis for image acquisition). It is also often desirable for probe 10 to remain fixed relative to the prostate 12 during biopsy or other treatment procedures such that the desired tissue locations may be targeted accurately.

To achieve such fixed positioning of probe 10, it is desirable to interface the probe 10 with a positioning device such as the exemplary positioning device 100 shown in FIG. 1B. The positioning device 100 maintains the probe 10 in a fixed position relative to the patient (e.g., prostate 12) and provides location information (e.g., frame of reference information) for use with an acquired image. In this regard, location outputs from the positioning device 100 may be supplied to a computer and/or imaging system 8. Likewise, the imaging output of the probe 10 may also be provided to the computer and/or imaging system 8, and the computer and/or imaging system may utilize this information to more accurately register the images 22 and display the tissue anatomy. Exemplary positioning devices are set forth in International Application No. PCT/CA2007/001076, entitled “APPARATUS FOR GUIDING A MEDICAL TOOL,” U.S. Pat. No. 7,832,114, entitled “TRACKER HOLDER ASSEMBLY,” and U.S. patent application Ser. No. 15/203,417 entitled “TRANSPERINEAL NEEDLE GUIDANCE,” the contents of which are fully incorporated herein by reference.

When attached to the positioning device 100, the probe handle is held by an arm of the positioning device having set of position sensors. These position sensors are connected to the computer of the imaging system via an embedded system interface. Hence, the computer has real-time information of the location and orientation of the probe 10 in reference to a unified rectangular or Cartesian (x, y, z) coordinate system. With the dimensions of the probe 10 taken into the calculations, the 3D orientations of the 2D image planes are known. The ultrasound probe 10 can send signals to the imaging system 8, which may be connected to the same computer (e.g., via a video image grabber) as the output of the position sensors. The imaging system therefore can generate real-time 2D images of the scanning area in memory. The image coordinate system and the arm coordinate system are unified by a transformation. Using the acquired 2D images, a prostate surface (e.g., 3D model of the organ) may be generated and displayed on a display screen in real-time.

The computer system runs application software and computer programs which can be used to control the system components, provide user interface, and provide the features of the imaging system. The software may be originally provided on computer-readable media, such as compact disks (CDs), magnetic tape, or other mass storage medium. Alternatively, the software may be downloaded from electronic links such as a host or vendor website. The software is installed onto the computer system hard drive and/or electronic memory and is accessed and controlled by the computer's operating system. Software updates may also be electronically available on mass storage media or downloadable from the host or vendor website. The software represents a computer program product usable with a programmable computer processor having computer-readable program code embodied therein. The software contains one or more programming modules, subroutines, computer links, and compilations of executable code, which perform at least some of the functions of the imaging system 8. The user may interact with the software via keyboard, mouse, voice recognition, and other user-interface devices (e.g., user I/O devices) connected to the computer system.

The 2D and/or 3D images may be used to plan for certain interventional procedures in which accuracy and/or precision is necessary to pinpoint a target site (e.g., biopsy, brachytherapy, cryo-ablation, etc.).

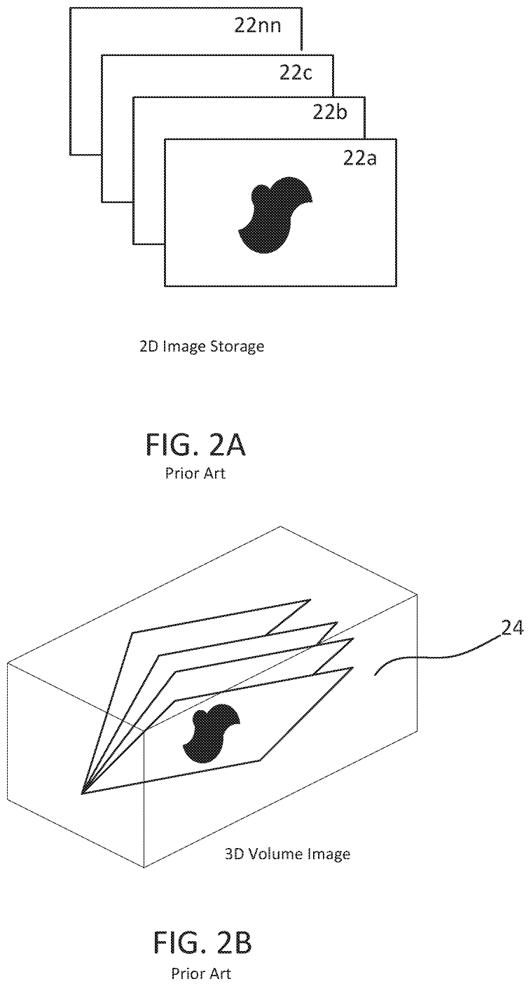

Turning to FIG. 2A, a plurality of 2D images 22a-22nn generated by the probe 10 may each be taken at a different angular position around the axis C-C′ of the probe 10 (as illustrated in FIG. 1B). As shown in FIG. 2B, these 2D images may be registered in a polar or cylindrical coordinate system. In such an instance, it may be beneficial for processing to translate images 22a-22nn into a rectangular coordinate system. In any case, 2D images 22a-22nn may be combined to generate a 3D image 24 which can be used to identify and target a location for intervention.

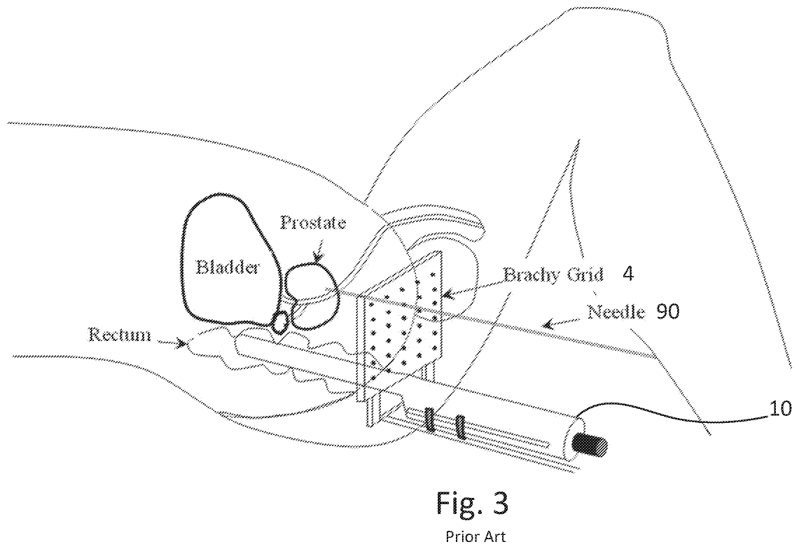

FIG. 3 illustrates a known intervention setup in which, with a patient in the lithotomic position, a side fire TRUS probe 10 is inserted into rectum of the patient while a tilting grid 4 (e.g., Brachy grid) is fixed relative to probe 10. A needle 90 containing, for example, brachytherapy seeds is inserted while being monitored on a display of the imaging system. The needle may be segmented to compute the insertion depth and deflection using real-time imaging. However, the method suffers from limitations. For instance, the method uses a traditional grid 4 for aligning the needle 90 with a target site. This limits the freedom of accessing a planned location to available grid locations and may be more problematic when there are anatomical obstructions (e.g. pelvic bone).

The utilities disclosed herein overcome the limitations of prior ultrasound guided biopsy and therapy systems by providing a combined probe holder for supporting a medical imaging instrument and needle guidance assembly that maintains a trajectory of an interventional needle held by the needle guidance assembly in a known positional relationship. Moreover, the known positional relationship may include an image plane of the medical imaging instrument held by a cradle of the probe holder. In this regard, the needle guidance assembly may be utilized, for example, to direct an interventional needle through a patient's perineum into the prostate to a target location while guided by a real-time display from the probe.

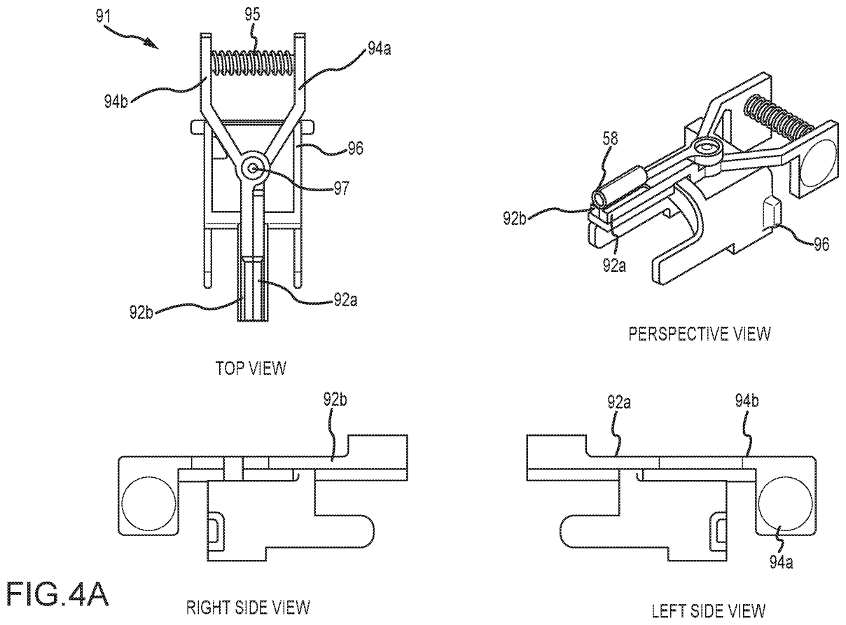

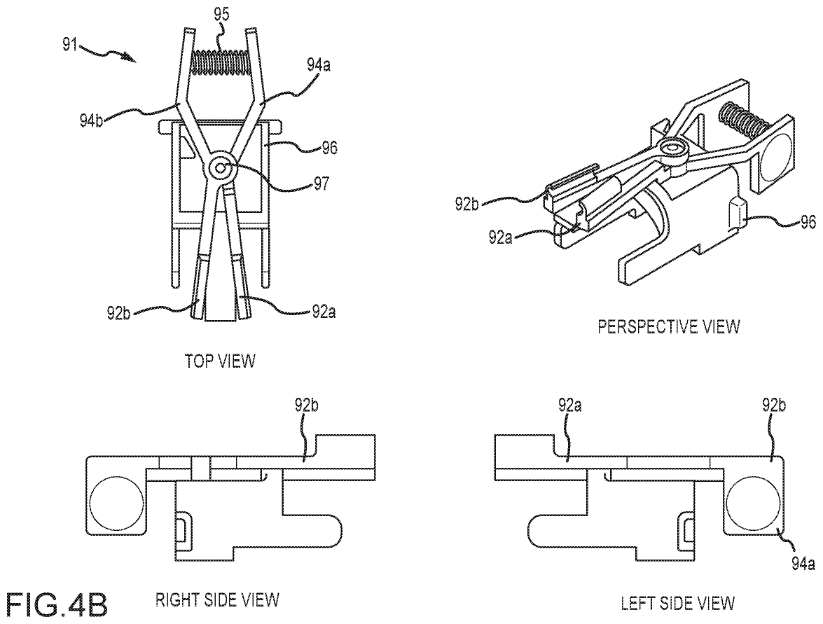

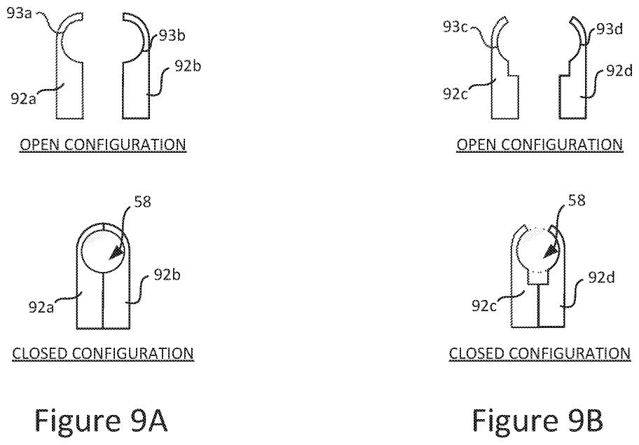

FIG. 4A illustrates several views of a needle guide 91 according to the present disclosure in a closed configuration. Needle guide 91 may be used in conjunction with a needle guidance assembly for restraining an interventional needle with respect to a medical imaging instrument. Needle guide 91 includes opposing lever arms which are interconnected and rotatable about a pivot pin 97. A left lever arm includes a left jaw 92a and left handle 94a extending therefrom. Similarly, a right lever arm includes a right jaw 92b and right handle 94b extending therefrom. Jaws 92a, 92b may each include a generally recessed surface such as a C-shaped channel (see FIGS. 9A-9B) extending along a portion thereof. In the closed configuration, these mirrored recessed channels may come together to form a fully enclosed guide bore 58 as in FIG. 9A or may be spaced apart to form an only partially enclosed guide bore as in FIG. 9B. Guide bore 58 may be sized and shaped to receive and constrain an interventional needle.

A biasing mechanism 95 may be used to bias the lever arms toward the closed configuration illustrated. Although illustrated as a coiled compression spring between the handles 94a, 94b, a biasing mechanism may additionally or alternatively include a tension spring, for example, disposed between the jaws 92a, 92b or may include a lever spring disposed adjacent to or around the pivot pin 97. Needle guide 91 may further include a mounting bracket 96 for associating the needle guide 91 with a base member of a needle guidance assembly or other device. In the illustrated embodiment, the mounting bracket 96 includes a generally U-shaped saddle configured to engage a base member and may include protrusions (not shown) from an inner surface of the saddle to engage corresponding recesses or grooves of the base member.

FIG. 4B illustrates the needle guide 91 of FIG. 4A but in an open configuration. In this arrangement, the compression spring forming the biasing mechanism 95 is under increased compression as may be caused by a user exerting opposing force on handles 94a, 94b. Release of the handles by the user will cause biasing mechanism 95 to return the needle guide 91 to the closed configuration. Although an interventional needle may be inserted through guide bore 58 in the closed configuration, such a process may risk damaging a distal end of such a needle or dislodging a therapeutic matter (e.g., Brachytherapy seed) from the distal end thereof. In other words, an interventional needle which is misaligned with the guide bore 58 by a user during insertion may impact a wall of jaws 92a, 92b causing complications or delay during a medical procedure. However, the split wall design of guide bore 58 (e.g., partial bore on right jaw 92a and corresponding partial bore on left jaw 92b) allows the guide bore 58 to be opened such that an interventional needle may be positioned within the opened bore and the needle guide 91 transitioned to the closed configuration around the interventional needle, thereby alleviating the risk of mishandling the needle. Moreover, the open configuration of needle guide 91 may improve the speed at which the interventional needle is engaged with the needle guidance assembly, thereby reducing the overall surgical procedure time. Furthermore, in some applications, confined spaces may introduce physical obstructions which may prevent a long interventional needle from being properly aligned and inserted into a guide bore. That is, there may be insufficient space available at the proximal end (opposite the patient) of a guide bore to allow a needle to be aligned and inserted into the guide bore due to, for example, a component of a positioning system of a medical imaging instrument. In this regard, the open configuration of needle guide 91 may permit a user to “drop-in” a needle from the top side of the needle guide 91 rather than needing to “slide” the needle through the guide bore from the rear.

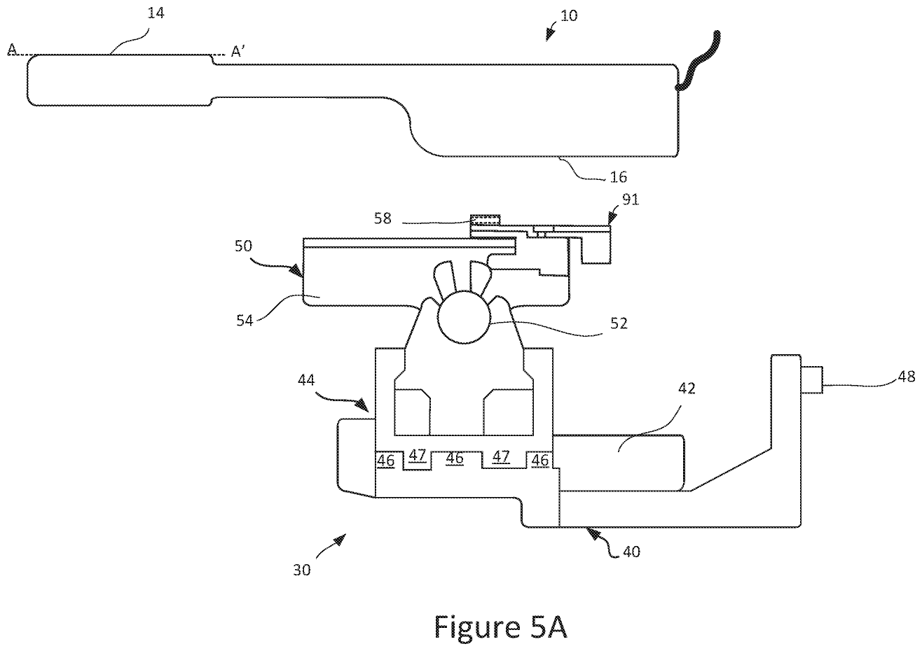

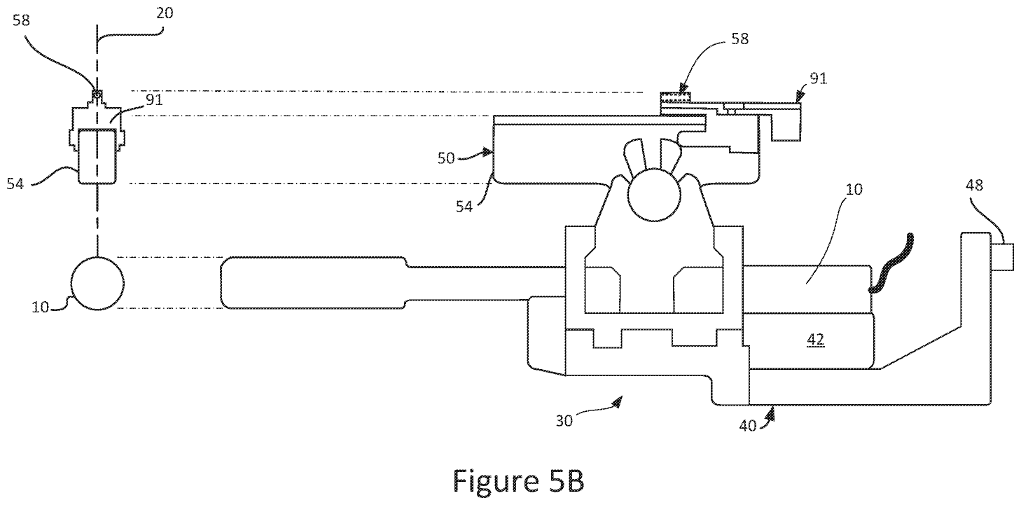

FIGS. 5A-5B illustrate one embodiment of a combined probe holder 40 and needle guidance assembly 50 (hereafter “cradle” 30). FIG. 5A illustrates the probe 10 outside of the cradle 30 and FIG. 5B illustrates the probe 10 disposed within the cradle 30. The cradle 30 may be used to interface ultrasound probe 10 with a positioning device (e.g., positioning device 100 of FIG. 1B, although any appropriate positioning device may be used). In the illustrated embodiment, the probe 10 includes an acquisition portion 14 defining an acquisition axis A-A′. The probe 10 also includes a handle portion 16 having a second length and a second diameter. Generally, the acquisition axis A-A′ and the handle 16 are offset such that they are nonaligned. However, that may not always be the case. The dimensions (e.g., lengths and/or diameters) of any or all of these components may vary between probes of different manufactures.

In the illustrated embodiment, the cradle 30 includes probe holder 40 having a recessed socket 42 that is sized to receive a handle portion 16 of the probe 10. Once the handle 16 of the probe 10 is located in the socket 42, the acquisition end 14 of the probe 10 extends beyond the distal end of the cradle 30 such that it may be inserted into a rectum of a patient. In the illustrated embodiment, the probe holder 40 includes a hinged clamp 44 that is connected to a first lateral edge of the recessed socket 42 via a plurality of mating knuckles 46, 47. A hinge pin (not shown) extends though these knuckles 46, 47. An opposing edge of the clamp 44 includes a latch (not shown) that allows for fixed attachment to an opposing later edge of the socket 42. In use, the clamp 44 is rotated open such that the handle 16 may be disposed within the socket 42. The clamp 44 may be rotated to a closed position and secured. This, in turn, secures the probe 10 within the socket 42 (see FIG. 5B).

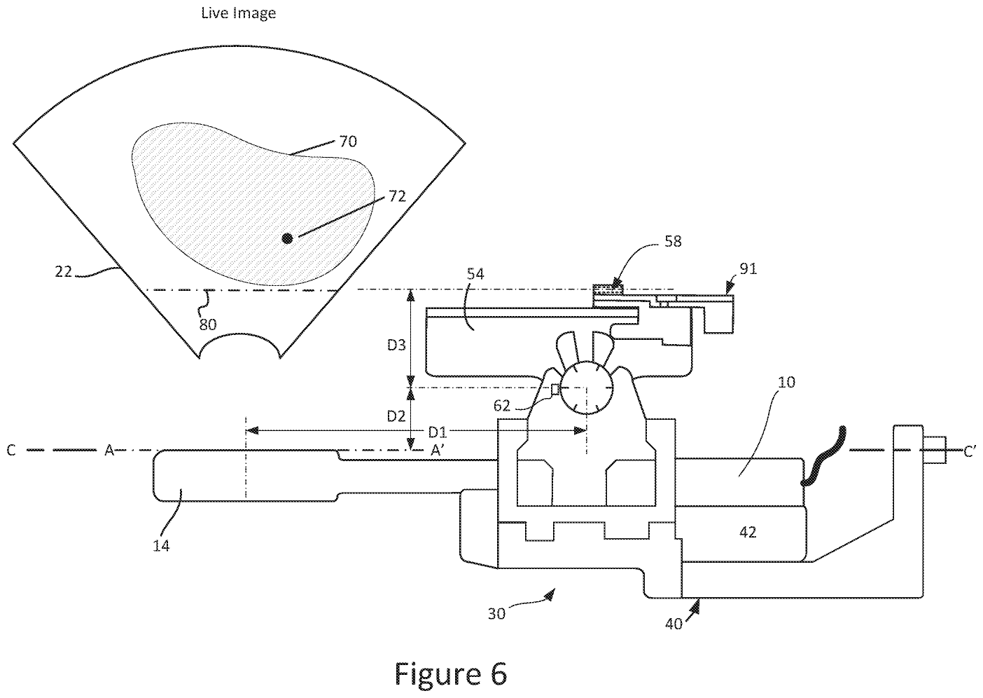

The socket 42 is a recessed surface that, in the present embodiment, is correspondingly shaped to the handle portion 16 of the ultrasound probe 10 such that the probe 10 may be disposed within the socket 42. Ultrasound probes from different OEMs may have differing shapes. In this regard, the socket 42 may include a deformable lining that allows for engaging differently configured probes. Alternatively, different sockets may be utilized for different probes. That is, the socket 42 may be removably connected (e.g., via bolts or screws) to the cradle 30 to allow matching a particular socket to a particular probe. In any arrangement, the acquisition axis A-A′ of the probe 10 may be aligned with a rotational axis C-C′ of the positioning device. See FIGS. 1B and 6. That is, the cradle 30 preferably, but not necessarily, interfaces with the positioning device such that an acquisition axis A-A′ of the probe 10 is aligned with a rotational axis C-C′ of the positioning device. This allows the acquisition portion 14 of the probe to rotate about a known fixed axis. Further, encoders of the positioning device may provide 3D location information allowing an image plane of the probe 10 to be identified in a 3D space. In the illustrated embodiment, the cradle 30 is connectable with the positioning device via a rotatable coupling 48 disposed at a proximal end of the cradle 30. This rotatable coupling 48 attaches to an arm of the positioning device and allows the cradle and supported probe 10 to rotate.

In addition to supporting probe holder 40, the cradle 30 also includes a needle guidance assembly 50 comprising a base member 54 and needle guide 91, which in the illustrated embodiment is fixedly connected to the clamp 44 which maintains the probe 10 within the socket 42. The needle guidance assembly 50 may be attached to other locations of the cradle 30 in other embodiments. As shown, the needle guidance assembly 50 is connected to an upper portion of the clamp 44 via an axle or spindle 52. The spindle 52 is received within a journal formed in the clamp 44. The spindle 52 also connects to an internal journal (not shown) in base member 54 of the needle guidance assembly 50. The spindle 52 permits the base member 54 of the needle guidance assembly 50 to rotate angularly relative to the probe holder 40 and supported probe 10. In one embodiment, the needle guidance assembly 50 rotates about an axis (e.g., center of spindle 52) that is transverse to an image plane of the probe 10 and/or the rotational axis of the positioning device. In such an embodiment, movement of the needle guidance assembly 50 and guide bore 58 is limited to one-degree of freedom within the image plane. Though discussed as using a spindle and journal, any hinged connection between the needle guidance assembly 50 and probe holder 40 may be utilized.

Removably connected to the base member 54 is a needle guide 91. The guide bore 58 of the needle guide 91 is sized to receive an interventional needle such that the interventional needle may selectively extend through the needle guidance assembly 50. The guide bore 58 of the needle guide 91 may be designed to accommodate various gauges of interventional needles. Alternatively, the needle guide 91 may be exchanged for other sized of needle guides to accommodate various interventional needles. In any case, an interventional needle may be extended through a distal forward surface of the needle guidance assembly 50.

The cradle 30 is designed such that the axis defined by the guide bore 58 of the needle guidance assembly 50 is aligned with the image plane of the supported probe 10. For instance, when a side fire ultrasound probe is utilized, an interventional needle extending through the guide bore 58 will extend into the image plane 20 of the ultrasound probe 10. That is, an axis or trajectory of the guide bore 58 is aligned within the image plane of the probe 10 as illustrated by the projection and front profile of the forward ends of the needle guidance assembly 50 and probe 10 in FIG. 5B.

Referring to FIG. 6, an exemplary image 22 of the ultrasound probe 10 as taken along the image plane is shown in relation to the cradle 30. As will be appreciated, in use, the live video or image 22 will be output on a display device. However, this image 22 is shown in relation to the cradle 30 for purposes of discussion. Rotational encoders 62 are connected to the spindle 52 such that the angular orientation of the base member 54 and guide bore 58 relative to probe holder 40 is known. That is, outputs from such encoders 62 may be provided to a computer for integration via the software to display the axis of the guide bore 58 (e.g., needle trajectory 80) on the image 22 provided by the probe 10. This is made possible by known dimensions of the systems such as D1 between the axis of rotation of the spindle and the center of the image plane, D2 between the acquisition axis A-A′ and the axis of rotation of the spindle, and D3 between the axis of rotation of the spindle and the axis of the guide bore 58.

During use for a prostate procedure, the ultrasound probe 10 may generate an image 22 including a representation of a patient's prostate 70. Further, due to the use of the encoders 62 between the base member 54 of the needle guidance assembly 50 and the probe holder 40, a needle trajectory 80 corresponding with an axis of the guide bore 58 may be calculated and displayed on the ultrasound image 22. That is, the known orientation of the needle guidance assembly 50 relative to the image plane of the ultrasound probe 10 allows for determining where an interventional needle extending through the guide bore 58 of the needle guidance assembly 50 will protrude into the image 22.

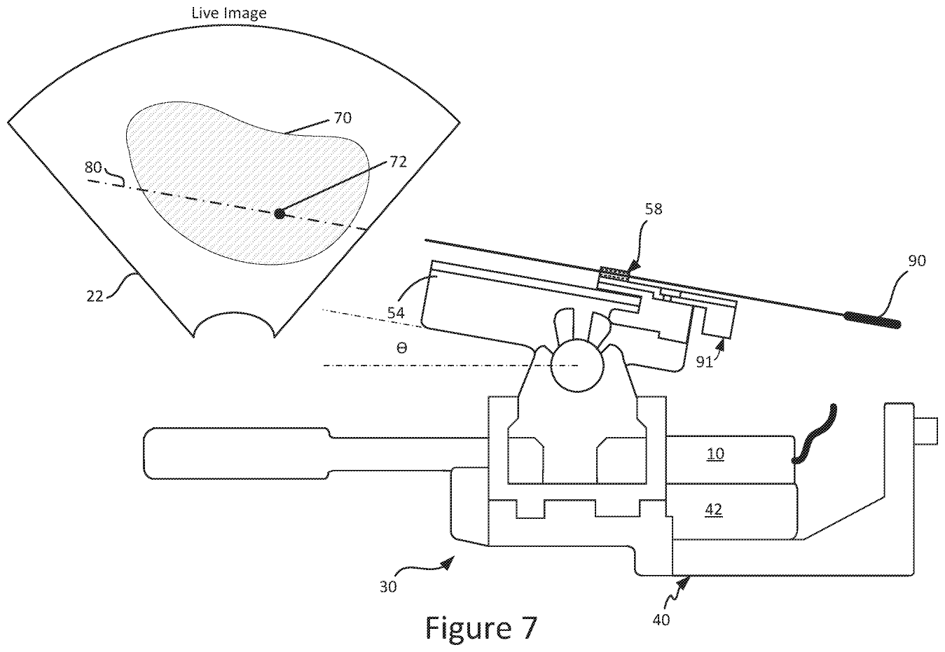

If there is a desired target site 72 within the image 22 (e.g., within the representation of a patient's prostate 70), the angular orientation Θ of the needle guidance assembly 50 may be adjusted about the spindle until the needle trajectory 80 intersects the target site 72 (as in FIG. 7). That is, as the angular orientation Θ of the needle guidance assembly 50 is adjusted with respect to the probe holder 40, the trajectory 80 displayed on the image 22 may be likewise adjusted. The angular orientation Θ of the needle guidance assembly 50 may be manually adjusted in one embodiment. In further embodiments, the angular orientation of the needle guidance assembly 50 may be robotically or electronically controlled. That is, various motors or other actuators may be utilized to align the needle trajectory 80 with the planned target site 72. In a further arrangement, the user may select a target site 72 on the ultrasound image 22 (e.g., via a touch screen or other user input) and the needle guidance assembly 50 may automatically align the trajectory 80 with the user selected target site 72.

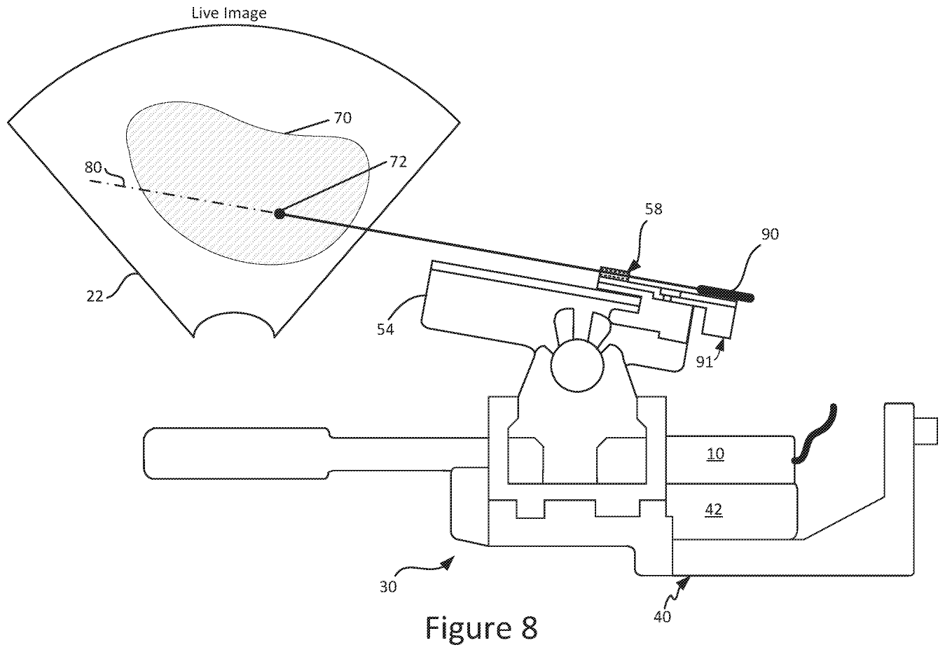

FIGS. 7 and 8 illustrate the disposition of an interventional needle 90 through the guide bore 58 of the needle guidance assembly 50. As will be appreciated, the interventional needle 90 may be disposed within the guide bore 58 of the needle guidance assembly 50 either prior to or after angular adjustment of the needle guidance assembly. In any case, once the guide bore 58 of the needle guidance assembly 50 is aligned such that the needle trajectory 80 extends through the target site 72, the interventional needle 90 may be advanced into the prostate to the target site 72. As the interventional needle 90 is disposed within the image plane of the ultrasound probe 10, the advancement of the interventional needle 90 into the prostate may be monitored in real time such that advancement may be terminated upon reaching the target site 72, as illustrated in FIG. 8. Once a biopsy is taken or therapy is applied to the target site 72, additional target sites may be biopsied and/or treated. This may entail rotating the cradle 30 to align the image plane with another target site. If appropriate, the initial interventional needle 90 may be removed from the needle guidance assembly 50 and replaced with another needle. That is, the needle guide 91 may be opened to remove the initial interventional needle from the needle guidance assembly 50 and to insert a different needle.

FIG. 9A illustrates a front view of a right jaw 92a and left jaw 92b, which include right recessed surface and a left recessed surface. In the illustrated embodiment these are formed as a right C-channel 93a and left C-channel 93b, respectively. In the open configuration, the jaws 92a, 92b are spaced apart such that an interventional needle may be disposed between the C-channels 93a, 93b. When transitioned to the closed configuration, the C-channels 93a, 9b form a fully enclosed guide bore 58. The guide bore 58 may be sized to correspond to a diameter of an intended interventional needle (e.g., same or similar diameter).

FIG. 9B illustrates a front view of a right jaw 92c and left jaw 92d, which include right C-channel 93c and left C-channel 93d, respectively. In the open configuration, the jaws 92c, 92d are spaced apart such that an interventional needle may be disposed between the C-channels 93c, 93d. When transitioned to the closed configuration, the C-channels 93c, 93d form a partially enclosed guide bore 58. In other words, because C-channels 93c, 93d do not extend a full 180-degrees (e.g., semi-circle), there is a space between the C-channels 93c, 93d even in the closed configuration.

In summary, the utilities disclosed herein allow for quick and safe placement of an interventional needle within a needle guidance assembly to facilitate real-time image guidance to targeted sites within an internal anatomical structure such as a patient's prostate. As a probe and needle guidance assembly are operative to co-rotate, any location within the anatomical structure may be imaged and targeted. Further, the ability to adjust the angular position of the needle guidance assembly within the plane of the ultrasound transducer likewise allows for targeting any location within the field of view of the imaging device.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described above are further intended to explain best modes known of practicing the invention and to enable others skilled in the art to utilize the invention in similar or other embodiments and with various modifications required by the particular application(s) or use(s) of the present invention. It is intended that the appended claims be construed to include alternative embodiments to the extent permitted by the prior art.

What is claimed is:

1. A needle guide for medical diagnoses and treatment, comprising:
first and second lever arms;
a pivot pin connecting the first and second lever arms and defining a pivot point between the first and second lever arms, wherein each of the first and second lever arms include a jaw and a handle extending from the jaw on an opposing side of the pivot point, wherein the first and second lever arms have a closed configuration in which the jaws are in physical contact and the handles are spaced apart by a maximum distance and an open configuration in which the jaws are spaced apart and the handles are spaced apart by less than the maximum distance; and
a guide bore configured to receive an interventional needle, wherein the guide bore is defined by the jaws when in the closed configuration.

2. The needle guide of claim 1, wherein the first lever arm includes a first recessed channel along the length of the jaw of the first lever arm and the second lever arm includes a second recessed channel along the length of the jaw of the second lever arm, wherein the first and second recessed channels form the guide bore when in the closed configuration.

3. The needle guide of claim 2, wherein the first and second recessed channels are contiguous in the closed configuration and completely enclose the guide bore.

4. The needle guide of claim 2, wherein at least a portion of the first and second recessed channels are spaced apart in the closed configuration such that a portion of the guide bore is unenclosed by the first and second recessed channels.

5. The needle guide of claim 1, further comprising:
a biasing mechanism configured to bias the first and second lever arms toward the closed configuration.

6. The needle guide of claim 2, wherein the biasing mechanism comprises a spring in compression disposed between the handle of the first lever arm and the handle of the second lever arm.

7. The needle guide of claim 6, wherein the biasing mechanism comprises a spring in tension disposed between the jaw of the first lever arm and the jaw of the second lever arm.

8. The needle guide of claim 1, further comprising:
a mounting bracket, wherein the first and second lever arms are affixed to the mounting bracket, and wherein the mounting bracket is configured for removable attachment to a base member of a system configured to hold a medical imaging instrument in pivotal relation to the base member.

9. The needle guide of claim 8, wherein at least one of the first and second lever arms is affixed to the mounting bracket via the pivot pin.

10. A system for medical diagnoses and treatment, comprising:
a probe holder configured to hold a medical imaging instrument;
an interventional needle; and
a needle guidance assembly, the needle guidance assembly comprising:
a base member pivotally attached to the probe holder for angular manipulation of the needle guidance assembly with respect to the medical imaging instrument; and
a needle guide, wherein the needle guide is removably attachable to the base member to retain the needle guide in fixed relation to the base member, the needle guide comprising:
first and second lever arms;
a pivot pin connecting the first and second lever arms and defining a pivot point between the first and second lever arms, wherein each of the first and second lever arms include a jaw and a handle extending from the jaw on an opposing side of the pivot point, wherein the first and second lever arms have a closed configuration in which the jaws are in physical contact and the handles are spaced apart by a maximum distance and an open configuration in which the jaws are spaced apart and the handles are spaced apart by less than the maximum distance; and
a guide bore configured to receive the interventional needle, wherein a trajectory axis of the guide bore is aligned within an image plane of the medical imaging instrument when the medical imaging instrument is disposed within the probe holder, wherein the guide bore is defined by the jaws when in the closed configuration.

11. The system of claim 10, wherein the medical imaging instrument is a side-fire ultrasound probe.

12. The system of claim 10, wherein the base member rotates about a second axis that is transverse to an image plane of the ultrasound probe.

13. The system of claim 10, wherein the interventional needle is a biopsy needle configured to extract tissue samples.

14. The system of claim 10, wherein the interventional needle is configured to deposit or apply therapeutic matter.

15. The system of claim 14, wherein the therapeutic matter comprises at least one of:
brachytherapy seeds;
a cryoablation fluid;
ablation energy; and
electroporation energy.

16. A method of administering treatment, comprising:
scanning a patient with a medical imaging instrument disposed in a probe holder;
identifying a target site within tissue of the patient;
aligning a guide bore of a needle guidance assembly with the target site, wherein the needle guidance assembly comprises:
a base member pivotally attached to the probe holder for angular manipulation of the needle guidance assembly with respect to the medical imaging instrument; and
a needle guide, wherein the needle guide is removably attachable to the base member to retain the needle guide in fixed relation to the base member, the needle guide comprising:
first and second lever arms;
a pivot pin connecting the first and second lever arms and defining a pivot point between the first and second lever arms, wherein each of the first and second lever arms include a jaw and a handle extending from the jaw on an opposing side of the pivot point, wherein the first and second lever arms have a closed configuration in which the jaws are in physical contact and the handles are spaced apart by a maximum distance and an open configuration in which the jaws are spaced apart and the handles are spaced apart by less than the maximum distance; and
the guide bore, wherein the guide bore is defined by the jaws when in the closed configuration; and
extending an interventional needle through the guide bore into the patient tissue to the target site.

17. The system of claim 16, wherein the interventional needle is a biopsy needle configured to extract tissue samples.

18. The system of claim 16, wherein the interventional needle is configured to deposit or apply therapeutic matter.

19. The system of claim 18, wherein the therapeutic matter comprises at least one of:
brachytherapy seeds;
a cryoablation fluid;
ablation energy; and
electroporation energy.
